(19) United States
(12) Patent Application Publication
(10) Pub. No.: US 2013/0085385 A1
(43) Pub. Date: Apr. 4, 2013

(54) SURGICAL LIGHTING SOURCES FOR USE WITH FLUOPHORE-TAGGED MONOCLONAL ANTIBODIES OR FLUOROPHORE-TAGGED TUMOR AVID COMPOUNDS

(71) Applicant: George A. Luiken, Coronado, CA (US)

(72) Inventor: George A. Luiken, Coronado, CA (US)

(21) Appl. No.: 13/477,895

(22) Filed: May 22, 2012

Related U.S. Application Data
(60) Provisional application No. 61/489,158, filed on May 23, 2011.

(51) Int. Cl. A61B 5/00; A61B 17/3205

(52) U.S. Cl. CPC A61B 5/0071; A61B 17/3205
USPC 600/431; 606/167

(57) ABSTRACT

The present invention describes light source devices to provide white and blue (401-510 nm) light for the in vivo identification of diseased tissue using fluorescence based tissue targeting. The light source devices are configured with a variety of LED lights capable of emitting white and blue light with at least one excitation wavelength in the range from about 401 nm to about 500 nm (for example, 470 nm to 495 nm) to irradiate an in vivo body part of a subject containing tumor or diseased tissue. The tumor or diseased tissue has fluorophore-tagged targeting constructs attached. The fluorophores used in the targeting constructs have emission spectra greater than 515 nm. The fluorescence emanating from the fluorescent targeting construct in response to the excitation wavelength is directly viewed with long-pass filtered (515 nm) lenses and is used to determine the location and/or surface area of the diseased tissue in the subject. Fluorescence based surgical identification provides more accurate disease resection.

### CROSS REFERENCE TO RELATED APPLICATION(S)

This application claims the benefit of priority under 35 U.S.C. §119(e) of U.S. Ser. No. 61/489,158, filed May 23, 2011, the entire contents of which is incorporated herein by reference in its entirety.

### BACKGROUND

1. Field of the Invention

The present invention relates generally to the medical field and more specifically to methods and devices for viewing the state of a body cavity or an internal organ of a mammalian body. The present invention relates to methods and devices more particularly blue light emitting diode (LED) surgical lighting systems for viewing the state of a body cavity or an internal organ of a mammalian body. More particularly, the invention relates to the light sources and methods used for detecting diseased or tumor tissue at an exterior or interior body site using an intravenously administered fluorescent targeting construct that binds to diseased tissue and which targeting construct is excited by light in the visible blue light range (401-510 nm). The ideal fluorophores for these fluorescent targeting constructs are fluorescein and fluorescein derivatives, with excitation spectra in the (blue) 470-495 nm range and maximum emission spectra close to 520 nm (green).

2. Background Information

Many solid and liquid substances naturally emit fluorescent radiation when irradiated with ultraviolet light or with near-infrared or the appropriate visible excitation light. However, the radiation may fall within wide wavelength bands of low and higher intensity. In the case of many natural objects, observations are partially obscured by natural fluorescence emanating simultaneously from many different compounds present in the sample under examination. In imaging devices such as microscopes, therefore, it is known to employ a filter for a selected UV or higher wavelength bands to screen out undesired fluorescence emanating from the object under observation.

In medical applications, a similar difficulty arises because both tumors and normal healthy tissue fluoresce naturally, albeit at different wavelengths. Consequently, when UV-activated fluorescence is used to detect tumors against a background of healthy tissue, identification of tumors is difficult. However, unlike most other cells of the body, tumor cells may possess a natural ability to concentrate and retain hematoporphyrin derivative dyes.

Based upon this discovery, a technique was developed wherein a hematoporphyrin derivative fluorescent dye is administered and allowed to concentrate in a tumor to be examined to increase the fluorescence from the tumor as compared with that of healthy background tissue. Hematoporphyrin dyes fluoresce within a fluorescence spectrum between 610 and 700 nm, a spectrum easy to detect. However, the natural fluorescence from healthy cells may be still much more intense than that from the dyes, and has a broader fluorescence spectrum. Thus, the use of fluorescent dyes in diagnosis of tumors had not been wholly successful. Recent work has demonstrated that using light in the visible blue range (401-510 nm) tumors and diseased tissue that has fluorophore-tagged monoclonal antibody (MAb) attached can be easily detected when viewing the tissue with a 515 nm (yellow) filter to block out the blue excitation light. (see references 1-6).

In endoscopic systems, it is also known to irradiate an internal organ with visible radiation to obtain a visible image and then to apply to the internal organ a fluorescent dye that concentrates in tumors over a period of time. The dye is allowed to concentrate, and then the internal organ is irradiated with excitation radiation for the dye to obtain a second fluorescent image. A body part having abnormal or diseased tissue, such as a cancer, may be identified by comparing an image produced by visible radiation of the internal organ with the image produced by fluorescence. To aid in visualizing the images received, endoscopic systems commonly utilize a video camera attached to a fiber optic scope having an optical guide fiber for guiding a beam from an external radiation source to the internal organ, and another optical guide fiber for transmitting a fluorescent image of the affected area to a television or LCD monitor for viewing.

These two approaches are combined in a method of the type disclosed in U.S. Pat. No. 4,821,117, wherein a fluorescent dye is applied to an object to be inspected, allowed to concentrate in the tumor, and the affected site is then alternately irradiated with visible light and with radiation at the excitation wavelength of the fluorophore. Images of the object obtained independently by visible and fluorescent light using a video camera are stored in memory, and are simultaneously displayed in a television monitor to visually distinguish the affected area of the body part from the healthy background tissue.

In another type of procedure, such as is described in U.S. Pat. No. 4,786,813, a beam-splitting system splits the fluorescence radiation passing though the optical system into at least three parts, each of which forms a respective image of the object corresponding to each of the wavelength regions received. A detector produces a cumulative weighted signal for each image point corresponding to a single point on the object. From the weighted signal values of the various points on the object, an image of the object having improved contrast is produced. This technique is used to aid in distinguishing the fluorescence from the affected tissue from that produced by normal tissue.

A still more complex method of visualizing images from an endoscopic device uses television scanning apparatus. For example, U.S. Pat. No. 4,719,508 discloses a method utilizing an endoscopic photographing apparatus wherein the endoscope includes an image sensor for successively generating image signals fed to a first frame memory for storing the image signals and a second frame memory for interlacing and storing image signals read successively from the first frame memory. The stored, interlaced image signals are delivered to a TV monitor for display to aid in visualizing the affected body part.

These endoscopic systems, which rely on photographic imaging of the area of interest (i.e. via a TV monitor), while effective, have historically relied on increasingly complex and expensive equipment and substitute image processing to construct a diagnostic image (i.e. indirect viewing) for direct viewing of the affected body part by the naked eye.

Certain of the fluorescent dyes that concentrate in tumors due to natural bodily processes can be excited at wavelengths corresponding to those produced by lasers to accomplish diagnostic and therapeutic purposes. Consequently, lasers have also been used in procedures utilizing endoscopic systems in conjunction with fluorescent dyes to image and treat tumors. In one embodiment of this general method, a dye is used that absorbs laser light at two different wavelengths and/or laser powers, one that excites fluorescence without generating damaging heat in the tissue, and one that generates sufficient heat in the dye to destroy surrounding tissue.

U.S. Pat. No. 4,768,513, for example, discloses a procedure in which a dye is applied to a body part suspected of containing a tumor, usually by local injection. The dye is allowed to concentrate in tumors and clear from healthy tissue over a period of days, and then the body part is irradiated with alternate pulses of two light sources: a white light of a known intensity and a fluorescence-exciting laser light. To compensate for variations in intensity of the fluorescence resulting from variations in the angle of incident light, and the like, visualization of the tumor is computer-enhanced by calculating the intensity of the fluorescence with respect to the known intensity of the white light. Ablation of a tumor detected using this method is accomplished by switching the laser to the heat-generating wavelength so as to destroy the cancerous tissue into which the fluorophore has collected.

While effective for diagnosing and treating tumor, such methods have two major drawbacks. Disease states other than tumor cannot be diagnosed, and laser visualization must be delayed for a period of two days or more after administration of the fluorescent dye to allow the dye to clear from normal tissue.

Monoclonal antibodies and other ligands specific for tumors and diseased tissue have been developed for use in diagnosis, both in tissue samples and in vivo. In addition to such ligands, certain tumor-avid and disease-avid moieties are disproportionately taken up (and/or optionally metabolized by tumor cells or diseased cells). Two well-known tumor-avid compounds are deoxyglucose, which plays a significant role in glycolysis in tumor cells, and somatostatin, which binds to and/or is taken up by somatostatin receptors in tumor cells, particularly in endocrine tumors. Other tumor-avid and disease-avid compounds i.e. methionine, histidine, folic acid, deoxy-galactose, cinacalcet, hormones, and porphyrin derivatives are also described.

In such studies, deoxyglucose is used as a radio-tagged moiety, such as fluorodeoxyglucose (¹⁸F-deoxyglucose), for detection of tumors of various types. It is believed that tumor cells experience such a mismatch between glucose consumption and glucose delivery that anaerobic glycolysis must be relied upon, thereby elevating the concentration of the radioactive tag in tumor tissue. It is also a possibility that the elevated concentration of deoxyglucose in malignant tumors may be caused by the presence of isoenzymes of hexokinase with abnormal affinities for native glucose or its analogs (A. Gjedde, Chapter 6: “Glucose Metabolism,” Principles of Nuclear Medicine, 2^{nd} Ed., W. B. Saunders Company, Philadelphia, Pa., pages 54-69). Similarly, due to the concentration of somatostatin in tumor tissue, radio-tagged somatostatin, and fragments or analogs thereof, are used in the art for non-invasive imaging of a variety of tumor types in a procedure known as somatostatin receptor scintigraphy (SRS).

Although these techniques have met with considerable success in determining the presence of tumor tissue, scintigraphic techniques are difficult to apply during a surgical procedure because of the equipment necessary for viewing the image provided by the radioisotope. Yet it is exactly at the time that the surgeon has made the incision or entered the body cavity that it would be most useful to “see” the outlines of the diseased tissue in real time, using “direct visualization” and without the need for expensive, highly technical, and time-consuming image processing equipment.

Thus there is a need in the art for simple, new and better methods and devices to directly visualize a broad range of putative disease sites without the need for use of image processing equipment.

### SUMMARY OF THE INVENTION

The need in the art for simple, new, and better methods to directly visualize a broad range of putative disease sites without the need for use of image processing equipment was addressed in U.S. Pat. Nos. 6,652,836, 6,299,860 and 6,284,223. The technology described by these patents and validated by in-vivo research utilizes fluorophores that have excitation in the blue 470-495 nm range and emission spectra in the green (515-525 nm) range. Ideal fluorophores for this technology are fluorescein and fluorescein derivatives. Fluorescein is a very safe molecule and its bright green emission color makes it very easily distinguished from any red, yellow and orange auto-fluorescence that can occasionally be seen. This technology for direct visualization whether through endoscopic devices or intra-operatively by the operating physician offers the additional advantage that the equipment required to view the disease tissue is comparatively simple and is less expensive that the equipment needed to process images. While digital imaging is possible with this methodology, because visible light (both white and blue 400-500 nm) is used, nothing other than yellow (515 nm) filtered goggles or lenses is needed to easily see the green fluorescence emanating from the diseased tissue. No processing equipment, i.e., charged couple device (CCD) is needed. The operating surgeon “sees” the green fluorescent diseased tissue and can accurately remove it directly and immediately.

The present disclosure addresses the light sources for use in illuminating fluorophore-tagged MAbs and fluorophore-tagged tumor-avid or fluorophore-tagged disease-avid compounds. These light sources allow the surgeon or operating physician to directly visualize all diseased tissue (i.e., cancer) and rapidly and accurately remove the diseased tissue at the time of resection. This direct viewing is made possible by using surgical operating room lighting devices (i.e. blue LED (401-510 nm) light sources) as described herein.

The present invention describes the light sources for illuminating or irradiating an in vivo body part of the subject containing diseased tissue with light having at least one excitation wavelength in the range from about 401 nm to about 500 nm or 510 nm. Fluorescence in response to the appropriate excitation wavelength emanating from a fluorescent targeting construct pre-administered to the subject and which has specifically bound to and/or been taken up by the diseased tissue in the body part, is directly viewed to determine the location and/or surface area of the diseased tissue in the subject and where indicated a portion or all of the diseased tissue is removed.

The location and/or surface area of the tumor tissue in the in vivo body part is diagnosed by administering a diagnostically effective amount of the targeting construct to the subject, allowing the targeting construct to bind to or be taken up by in vivo tumor cells or other diseased cells, and directly viewing fluorescence emanating from the targeting construct bound to or taken up in the tumor tissue or diseased tissue in response to irradiation of the tumor tissue with a light that provides the required excitation wavelength.

The light sources may all include a plurality or mixture of alternate sources of visible white and visible blue (400-500 nm) light (typically blue light emitting diodes (LEDs)) with the blue light sources having the capability of being further filtered with band-pass filters to narrow the excitation wavelength to about 470-495 nm.

### BRIEF DESCRIPTION OF THE DRAWINGS

FIG. 1 is a diagram of a device in an embodiment of the invention. The device includes blue LED capability with some or all of the lights being blue LEDs (401-510 nm (with or without band-pass filters (i.e. 470-495 nm). Lights may be fixed to the ceiling or walls and include movable connecting arms or may be on mobile bases. Each lighting device may be mechanically, motion or voice activated.

FIG. 2 is a diagram of a device in an embodiment of the invention.

FIG. 3 is a diagram of a device in an embodiment of the invention.

FIG. 4 is a bottom view of the magnification lens frame of the device depicted in FIG. 3 in an embodiment of the invention.

FIG. 5 is a diagram of a device in an embodiment of the invention.

FIG. 6 is a diagram of a device in an embodiment of the invention.

### DETAILED DESCRIPTION OF THE INVENTION

The present invention describes devices for in vivo identification of diseased tissue in a subject in need thereof. The invention includes a variety of light sources for irradiating an in vivo body part of the subject containing tumor tissue or other diseased tissue with light having at least one excitation wavelength in the range from about 401 nm to about 500 nm. Fluorescence emanating from a fluorescent targeting construct administered to the subject and which has specifically bound to and/or been taken up by the diseased tissue in the body part, in response to the at least one excitation wavelength (i.e., 470-495 nm range) is directly viewed to determine the location and/or surface area of the diseased tissue in the subject.

The devices and methods described herein relate to and are intended for use with fluorescence imaging technology as described in U.S. Pat. Nos. 6,652,836, 6,299,860 and 6,284,223, all titled “Method For Viewing Diseased Tissue Located Within A Body Cavity,” each of which is incorporated herein by reference in its entirety.

Before the present devices and methods are described in further detail, it is to be understood that this invention is not limited to particular devices and methods described, as such devices and methods may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

Light having a wavelength range from 401 nm to 500 nm lies within the visible range of the spectrum, in contrast to UV light, which lies within the range from about 4 nm to about 400 nm and near infrared (NIR) which lies in the non-visible range from 750-1200 nm. Therefore, the excitation light sources used in practice of the invention diagnostic methods will provide at least one wavelength of light that illuminates surrounding tissue as well as exciting fluorescence from the fluorescent targeting construct used in practice of the invention methods (i.e. in the 470-495 nm range). The excitation light may be monochromatic or polychromatic. To compensate for the tendency of such background effect of the excitation light source (for example blue LEDs) to obscure the desired diagnostic image, it is preferred to use a filter to screen out wavelengths below about 515 nm in the excitation light, thereby eliminating wavelengths that would be reflected from healthy tissue so as to cause loss of resolution of the fluorescent image.

In various embodiments of the invention, band-pass filters may be used on the excitation light source (blue LEDs for example) to filter or screen out all wavelengths of light except those in a narrow band (i.e. 470-495 nm). Likewise long-pass filters (i.e. 515 nm) may be used for viewing (i.e. goggles, endoscopes, cameras, microscopes, magnifying lenses and the like) during the diagnostic procedure or surgical procedure. The filter may be a polarizing filter or a non-polarizing filter. For example, a yellow filter will generally filter out light below 515 nm, thus eliminating the blue excitation light and allowing the emission light (emanating from the fluorescent targeting construct) to be seen.

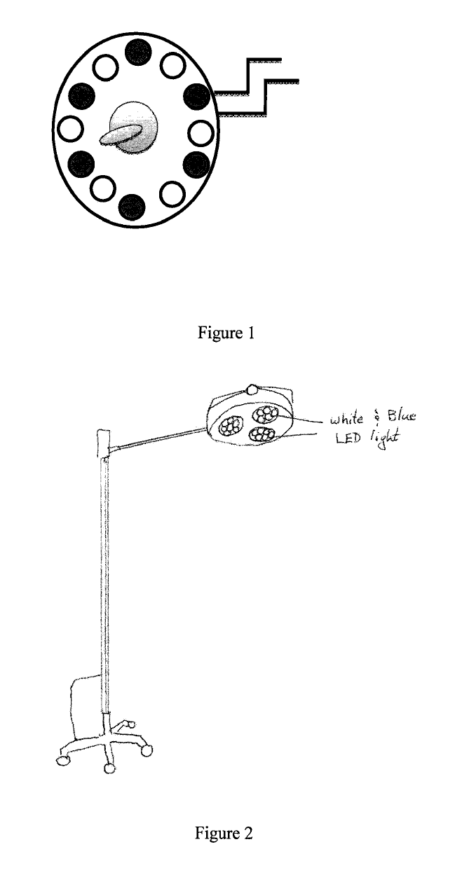

Operating rooms can be equipped with overhead surgical lighting devices of the present invention that produce wavelengths of light in the optical emitting spectrum useful in practice of invention. Such overhead lighting devices as described herein could include an array or mixture of white light sources as well as an array or mixture of blue LED (400-500 nm) light sources or be composed of all blue LEDs. (See FIG. 1) Each blue LED light could be fitted (in an exemplary embodiment) with a band-pass filter (470-495 nm) for optimum excitation of the fluorophores that would typically be used (i.e. fluorescein, fluorescein derivatives, Alexa Fluor 488, Hy-Lyte 488, and the like). A light may be utilized in the practice of the invention merely by dimming or turning out the other lights in the operating room (to reduce or eliminate extraneous light that would be visibly reflected from tissue in the body part under investigation) and shining the excitation light into the body cavity or surgically created opening so that the fluorescent image received (using long-pass filtering (i.e. yellow filtered lenses)) directly by the eye of the observer (e.g., the surgeon) is predominantly the fluorescent image emanating from the fluorescent targeting construct.

In various embodiments, overhead light sources of the present invention may have the capability as with most overhead operating room light sources to be fixed to the ceiling with movable arms for adjustment of height or angle, movable on wheels, be attached to flexible arms, fitted with cameras for image capture, voice controlled and of varying intensities of lumen output, depending on need.

Light in the 401 nm to 500 nm wavelength range is readily absorbed in tissue. Accordingly, in invention diagnostic methods, the diseased tissue (and bound or taken-up targeting construct) is “exposed” to the excitation light (e.g, by surgically created opening or endoscopic delivery of the light to an interior location. The invention method is particularly suited to in vivo detection of diseased tissue located at an interior or exterior site in the subject, such as within a natural body cavity or a surgically created opening, where the diseased tissue is “in plain view” (i.e., exposed to the human eye) to facilitate a procedure of biopsy or surgical excision. As the precise location and/or surface area of the tumor tissue is readily determined by the invention diagnostic procedure, the invention method is a valuable guide to the surgeon, who needs to “see” in real time the exact outlines, size, and the like of the mass to be resected as the surgery proceeds without relying on a capture device.

If the putative diseased site is a natural body cavity or surgically produced interior site, an endoscopic device can be optionally used to deliver the excitation light to the site, to receive fluorescence emanating from the site within a body cavity, and to aid in viewing of a direct image of the fluorescence from the diseased tissue. For example, a lens in the endoscopic device can be used to focus the detected fluorescence as an aid in formation of the image. As used herein, such endoscope-delivered fluorescence is said to be “directly viewed” by the practitioner and the tissue to which the targeting construct binds or in which it is taken up must be “in plain view” to the endoscope since the light used in the invention diagnostic procedure will not contain wavelengths of light that penetrate tissue, such as wavelengths in the near infra red range. Alternatively, as described above, the excitation light may be directed by any convenient means into a body cavity or surgical opening containing a targeting construct administered as described herein and the fluorescent image so produced can be directly visualized by the eye of the observer with or without aid of an endoscope. Direct viewing in this invention means that the fluorescent image produced by the invention method is such that it can be viewed without aid of an image processing device, such as a CCD camera, TV monitor, photon collecting device, and the like. This ability to view the image directly is important in that it eliminates the need for having a capture device at any time during a surgical procedure.

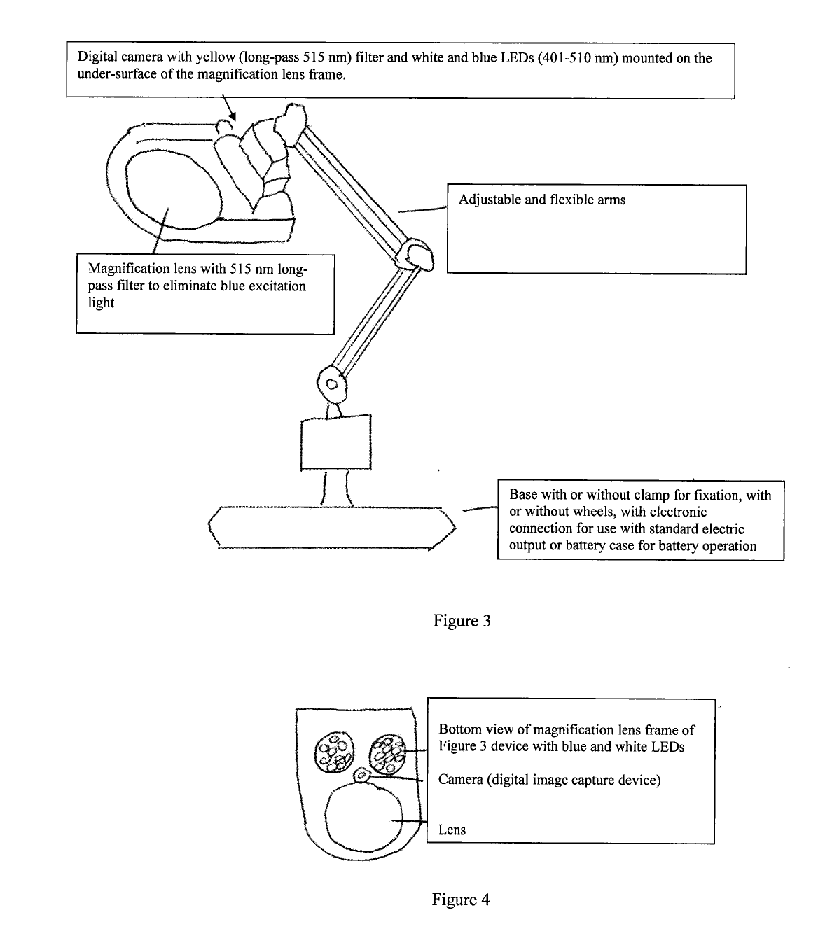

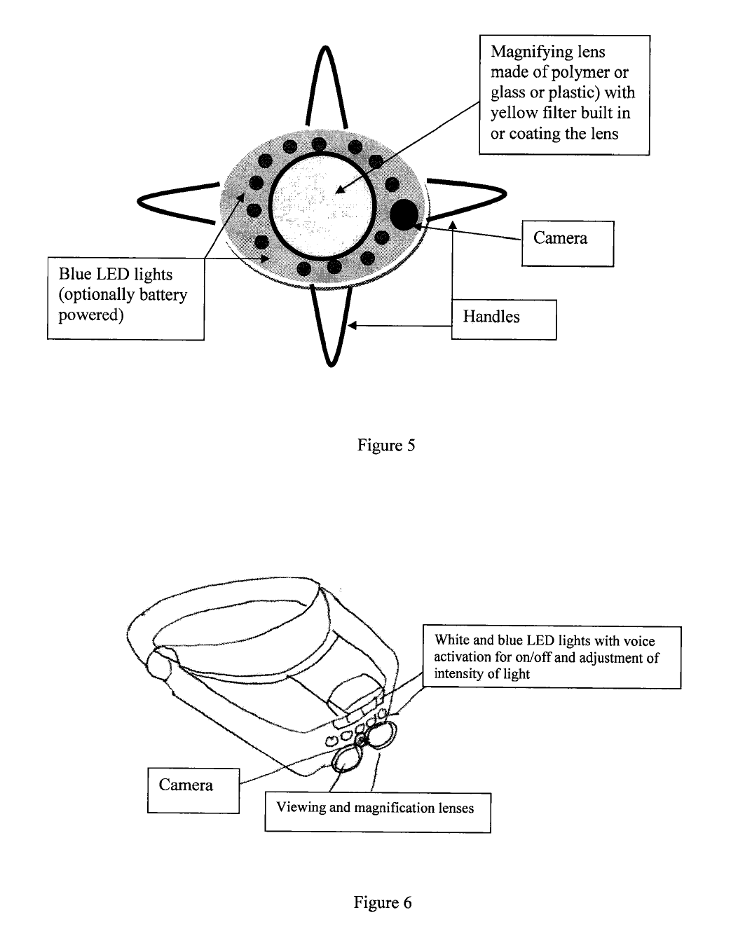

In one embodiment of the invention, the light source may be one used in an examination or operating room and can be hand held movable, fixed to an examination table, have a flexible or fixed arm and be fitted with a magnifying lens with removable yellow filter (515 nm) (see FIGS. 2, 3, 4 and 5).

In another embodiment of the invention, the light source may be one worn by the surgeon or physician on the head (see FIG. 6) which would provide white and blue (400-500 nm) light and could also include a camera for image capture, magnifying lenses or protective lenses and be fitted with yellow filters which could be moved into or out of the field of view.

In one embodiment, the light source may be micro white and blue LEDs (401-510 nm) at the distal viewing end of an endoscopic device where the camera if fitted at the end of the endoscopic device.

In one embodiment of the invention diagnostic methods, diseased or abnormal tissue is contemporaneously viewed through a surgical opening to facilitate a procedure of biopsy or surgical excision. As the location and/or surface area of the diseased tissue are readily determined by the invention diagnostic procedure, the invention method is a valuable guide to the surgeon, who needs to know the exact outlines, size, etc. of the mass, for example, for resection as the surgery proceeds.

Accordingly, in this embodiment, the present invention provides devices with and methods for light sources used in a diagnostic procedure during surgery in a subject in need thereof by irradiating an in vivo body part of the subject containing diseased tissue with light having at least one excitation wavelength in the range from about 401 nm to about 510 nm, directly viewing fluorescence emanating from a targeting construct administered to the subject that has specifically bound to and/or been taken up by the diseased tissue in the body part, wherein the targeting construct fluoresces in response to at least one excitation wavelength (preferably 470-495 nm), determining the location and/or surface area of the diseased tissue in the subject, and when necessary, removing at least a portion of the diseased or tumor tissue.

In one embodiment of the invention method, a single type of fluorescent moiety is relied upon for generating fluorescence emanating from the irradiated body part (i.e., from the fluorescent targeting construct that binds to or is taken up by diseased tissue). Since certain types of healthy tissue fluoresce naturally, in such a case it is important to select a fluorescent moiety for the targeting construct that has a predominant excitation wavelength that does not contain sufficient wavelengths in the visible range of light to make visible the surrounding healthy tissue and thus inhibit resolution of the diseased tissue. Therefore, the light source used in practice of this embodiment of the invention provides light in the range from about 401 nm to about 500 nm (preferably 470-495 nm) as the excitation source and the fluorophores used in this invention have an emission spectra in the 515 nm and greater.

However, if a combination of targeting ligands that fluoresce at different wavelengths is used in practice of the invention, the spectrum of the excitation light must be broad enough to provide at least one excitation wavelength for each of the fluorophores used. For example, it is particularly important when fluorophores of different colors are selected to distinguish one type of diseased tissue from another type of diseased tissue, that the excitation spectrum of the light(s) include excitation wavelengths for the fluorophores targeted both types of diseased tissue.

Additional non-limiting examples of fluorescent compounds that fluoresce in response to an excitation wavelength in the range from 401 nm to about 500 nm are found in Table 1 below.

TABLE 1

| | EXCITATION | EMISSION |
|---|---|---|
| COMPOUND | RANGE (nm) | RANGE (nm) |
| Acridine Red | 455-600 | 560-680 |
| Acridine Yellow | 470 | 550 |
| Acriflavin | 436 | 520 |
| AFA (Acriflavin Feulgen SITSA) | 355-425 | 460 |
| ACMA | 430 | 474 |
| Astrazon Orange R | 470 | 540 |
| Astrazon Yellow 7 GLL | 450 | 480 |
| Atabrine | 436 | 490 |
| Auramine | 460 | 550 |
| Aurophosphine | 450-490 | 515 |
| Aurophosphine G | 450 | 580 |
| Berberine Sulphate | 430 | 550 |
| BOBO-1, BO-PRO-1 | 462 | 481 |
| BOPRO 1 | 462 | 481 |
| Brilliant Sulpho-flavin FF | 430 | 520 |
| Calcein | 494 | 517 |
| Calcofluor White | 440 | 500-520 |
| Cascade Blue | 400 | 425 |
| Catecholamine | 410 | 470 |
| Chinacrine | 450-490 | 515 |
| Coriphosphine O | 460 | 575 |
| DiA | 456 | 590 |
| Di-8-ANEPPS | 488 | 605 |
| DiO [DiOC₁₈(3)] | 484 | 501 |
| Diphenyl Brilliant Flavine 7GFF | 430 | 520 |
| Euchrysin | 430 | 540 |
| Fluorescein | 494 | 518 |
| Fluorescein Iso-thiocyanate (FITC) | 490 | 525 |
| Fluo 3 | 485 | 503 |
| FM1-43 | 479 | 598 |
| Fura Red | 472 (low [Ca²⁺]) | 657 (low [Ca²⁺]) |
| | 436 (high [Ca²⁺]) | 637 (high [Ca²⁺]) |
| Genacryl Brilliant Yellow 10GF | 430 | 485 |
| Genacryl Pink 3G | 470 | 583 |
| Genacryl Yellow SGF | 430 | 475 |
| Gloxalic Acid | 405 | 460 |
| 3-Hydroxypyrene- | 403 | 513 |
| 5,-8,10-TriSulfonic Acid | | |
| 7-Hydroxy-4-methylcourmarin | 360 | 455 |
| 5-Hydroxy-Tryptamine (5-HT) | 380-415 | 520-530 |
| Lucifer Yellow CH | 425 | 528 |
| Lucifer Yellow VS | 430 | 535 |
| LysoSensor Green DND-153, | 442 | 505 |
| DND-189 | | |
| Maxilon Brilliant Flavin 10 GFF | 450 | 495 |
| Maxilon Brilliant Flavin 8 GFF | 460 | 495 |
| Mitotracker Green FM | 490 | 516 |
| Mithramycin | 450 | 570 |
| NBD | 465 | 535 |
| NBD Amine | 450 | 530 |
| Nitrobenzoxadidole | 460-470 | 510-650 |
| Nylosan Brilliant Flavin E8G | 460 | 510 |
| Oregon Green 488 fluorophore | 496 | 524 |
| Phosphine 3R | 465 | 565 |
| Quinacrine Mustard | 423 | 503 |
| Rhodamine 110 | 496 | 520 |
| Rhodamine 5 GLD | 470 | 565 |
| Rhodol Green fluorophore | 499 | 525 |
| Sevron Orange | 440 | 530 |
| Sevron Yellow L | 430 | 490 |
| SITS (Primuline) | 395-425 | 450 |
| Sulpho Rhodamine G Extra | 470 | 570 |
| SYTO Green fluorescent | 494 ± 6 | 515 ± 7 |
| nucleic acid stains | | |
| Thioflavin S | 430 | 550 |
| Thioflavin 5 | 430 | 550 |
| Thiozol Orange | 453 | 480 |
| Uranine B | 420 | 520 |
| YOYO-1, YOYO-PRO-1 | 491 | 509 |

Since the fluorescence properties of biologically compatible fluorophores are well known, or can be readily determined by those of skill in the art, the skilled practitioner can readily select a useful fluorophore or useful combination of fluorophores, and match the wavelength(s) of the excitation light to the fluorophore(s). Toxicity of additional useful fluorophores can be determined using animal studies as known in the art.

Preferably, the targeting construct (e.g., the ligand moiety of the invention targeting construct) is selected to bind to and/or be taken up specifically by the target tissue of interest, for example to an antigen or other surface feature contained on or within a cell that characterizes a disease or abnormal state in the target tissue.

In one embodiment according to the present invention, the disease or abnormal state detected by the invention method can be any type characterized by the presence of a known target tissue for which a specific binding ligand is known. For example, various heart conditions are characterized by production of necrotic or ischemic tissue or production of atherosclerotic tissue for which specific binding ligands are known. As another illustrative example, breast cancer is characterized by, but not limited to the production of tumor antigens or specific receptor molecules identified by monoclonal antibodies (i.e. CA15-3, CA19-9, CEA, or HER2/neu, estrogen receptor proteins, progesterone receptor proteins). It is contemplated that the target tissue may be characterized by cells that produce either a surface antigen for which a binding ligand is known, or an intracellular marker (i.e. antigen), since many targeting constructs penetrate the cell membrane.

Representative disease states that can be identified using the invention method include such various conditions as different types of tumors, bacterial, fungal and viral infections, inflammation, and the like. As used herein “abnormal” tissue can include but is not limited to cancer, precancerous conditions, necrotic or ischemic tissue, and tissue associated with connective tissue diseases, and auto-immune disorders, inflammation and the like. Further, examples of the types of target tissue suitable for diagnosis or examination using the invention method include cardiac disease, inflammatory arterial plaques, and cancer of the breast, ovary, uterus, lung, endothelial, vascular, esophagus, stomach, colon, rectum, small intestine, prostate, bladder, kidney, thyroid, lung, head and neck, parathyroid, liver, pancreas, adrenal glands, brain, endocrine tissue, and the like, as well as combinations of any two or more thereof.

The targeting construct is administered in a “diagnostically effective amount.” An effective amount is the quantity of a targeting construct necessary to aid in direct visualization of any target tissue located in the body part under investigation in a subject. A “subject” as the term is used herein is contemplated to include any mammal, such as a domesticated pet, farm animal, or zoo animal, but preferably is a human. Amounts effective for diagnostic use will, of course, depend on the size and location of the body part to be investigated, the affinity of the targeting construct for the target tissue, the type of target tissue, as well as the route of administration. Local administration of the targeting construct will typically require a smaller dosage than any mode of systemic administration, although the local concentration of the targeting construct may, in some cases, be higher following local administration than can be achieved with safety upon systemic administration.

Since individual subjects may present a wide variation in severity of symptoms and each targeting construct has its unique diagnostic characteristics, including, affinity of the targeting construct for the target, rate of clearance of the targeting construct by bodily processes, the properties of the fluorophore contained therein, and the like, the skilled practitioner will weigh the factors and vary the dosages accordingly.

The invention fluorescing targeting constructs can be produced by well known techniques. For example, well known techniques of protein synthesis can be used to obtain proteinaceous components of the targeting construct if the amino acid sequence of the component is known, or the sequence can first be determined by well known methods, if necessary. Some of the ligand genes are now commercially available.

Some abbreviations used herein include:

MAb (monoclonal antibody);

CEA (carcinoembryonic antigen);

CA15-3 (cancer antigen 15-3);

HER2 (human epidermal growth factor receptor 2);

LED (light emitting diode); and

nm (nanometer).

### REFERENCES AND PATENTS

1. Billinton, Nicholas and Knight, Andrew W. (2001). Seeing the Wood through the Trees: A Review of Techniques for Distinguishing Green Fluorescent Protein from Endogenous Autofluorescence. Analytical Biochemistry 291, 175-197.
2. Andersson, H., et al; Autofluorescence of living cells. Journal of Microscopy, Vol. 191, Pt 1, July 1998, pp. 1-7.
3. Kessel, D; HEMATOPORPHYRIN and HPD: PHOTOPHYSICS, PHOTOCHEMISTRY and PHOTOTHERAPY, Photochemistry and Photobiology; Volume 39, Issue Supplement s1, pages 851-859, May 1984.
4. Cao H S, Kaushal S, Metildi C A, Menen R S, Lee C, Snyder C S, Messer K, Pu M, Luiken G A, Talamini M A, Hoffman R M, Bouvet M. Tumor-Specific Fluorescence Antibody Imaging Enables Accurate Staging Laparoscopy in an Orthotopic Model of Pancreatic Cancer. Hepatogastroenterology. 2012 Jan. 11; 59(118). doi: 10.5754/hge11836. [Epub ahead of print].
5. Kaushal S, McElroy M K, Luiken G A, Talamini M A, Moossa A R, Hoffman R M, Bouvet M. Fluorophore-conjugated anti-CEA antibody for the intraoperative imaging of pancreatic and colorectal cancer. J Gastrointest Surg. 2008 November; 12(11):1938-50. Epub 2008 Jul. 30.
6. McElroy M, Kaushal S, Luiken G A, Talamini M A, Moossa A R, Hoffman R M, Bouvet M. Imaging of primary and metastatic pancreatic cancer using a fluorophore-conjugated anti-CA19-9 antibody for surgical navigation. World J. Surg. 2008 June; 32(6):1057-66.
7. Johnson T E, Luiken G A, Quigley M M, Xu M, Hoffman R M. In vivo fluorescence of medullary carcinoma of the thyroid: a technology with potential to improve visualization of malignant tissue at surgical resection. Ear Nose Throat J. 2008 August; 87(8):E1.
U.S. Patents describing a variety of surgical lighting systems include: U.S. Pat. Nos. 5,580,163; 5,274,535; 5,093,769; 4,608,622; 4,316,237; 4,651,257; 4,380,794; 4,288,844; 4,254,454; 4,630,182; 3,702,928; 2,280,402; and 2,069,950.

Although the invention has been described with reference to the above embodiments, it will be understood that modifications and variations are encompassed within the spirit and scope of the invention. Accordingly, the invention is limited only by the following claims.

What is claimed is:

1. A method for in vivo diagnosis of diseased tissue in a subject in need thereof, the method comprising:
a) providing a light source device comprising one or more light sources configured to provide at least one excitation wavelength in the range from about 401 nm to about 510 nm or from about 470 nm to about 495 nm;
b) irradiating an in vivo body part of the subject containing diseased tissue with the light source and directly viewing fluorescence emitted in response to the light source, the fluorescence being emitted from a fluorescent targeting construct administered to the subject and which is specifically bound to or taken up by the diseased tissue in the body part; and
c) determining a location or a surface area of the diseased tissue in the subject from the fluorescence provided by the targeting construct.

2. The method of claim 1, wherein the light is substantially lacking in wavelengths greater than about 500 nm or 510 nm.

3. The method of claim 1, wherein the light source device provides visible blue light of from about 401 nm to about 500 nm, and wherein the visible blue light is substantially monochromatic.

4. The method of claim 3, wherein the wavelength of the visible blue light is matched to a predominant excitation wavelength of the fluorescent targeting construct.

5. The method of claim 4, wherein the predominant excitation wavelength of the fluorescent targeting construct is about 470 nm to about 495 nm and the wavelength of the visible blue light is about 470 nm to about 495 nm.

6. The method of claim 3, further comprising providing white light in combination with the blue light, the white light being emitted from the one or more additional light sources.

7. The method of claim 6, wherein the white light and the blue light is emitted from an array of light sources comprising a combination of light sources configured to emit the white light and the blue light.

8. The method of claim 1, wherein the light source device is configured as an overhead light source, the light source device comprising both white and blue light sources for illuminating an operative field.

9. The method of claim 3, wherein each blue light source comprises a band-pass filter of about 470 nm to about 495 nm.

10. The method of claim 1, wherein light source device further comprises a digital imaging device for capturing images during a surgical procedure.

11. The method of claim 10, wherein the light source device further comprises functionality for directional, mechanical or voice activated positioning.

12. The method of claim 10, wherein the digital imaging device comprises functionality for long-pass filtering of about 515 nm.

13. The method of claim 12, wherein the digital imaging device further comprises zoom magnification lenses.

14. The method of claim 1, wherein the light source device is an extendable fiberoptic light comprising a light source for white light and blue light.

15. The method of claim 1, wherein the one or more light sources comprise functionality for variable light intensity output.

16. The method of claim 1, wherein the light source device is configured as a hand-held device.

17. The method of claim 16, wherein the light source device further comprises a magnifying lens having a removable yellow filter of about 515 nm.

18. The method of claim 17, wherein the light source device further comprises a digital imaging device.

19. The method of claim 18, where the light source device is coupled to an examination headlamp.

20. The method of claim 19, wherein the light source device further comprises one or more of both white light and blue light sources, a digital imaging device, one or more magnification lenses, and a removable yellow filter of about 515 nm.

21. The method of claim 1, wherein the targeting construct comprises a biologically compatible fluorescing moiety responsive to the excitation wavelength and a targeting ligand moiety selected from a monoclonal antibody or partial antibody or combination thereof, tumor-avid moiety, disease-avid moiety, hormone, hormone-receptor binding peptide, deoxyglucose, doxygalactose, methionine, folic acid, histidine, somatostatin, a somatostatin receptor-binding peptide, cinacalcet, or any combination thereof.

22. The method of claim 1, wherein the diseased tissue is associated with a condition selected from the group consisting of benign or malignant tumors, bacterial, fungal and viral infections, pre-cancerous conditions, heart attack, stroke, and necrotic, inflammatory and ischemic conditions.

23. The method of claim 1, further comprising surgically excising at least a part of the diseased tissue while directly viewing the fluorescence pattern.

24. The method of claim 1, wherein the surface area is determined by the intensity of the emitted fluorescence as compared with background fluorescence intensity.

25. The method of claim 1, wherein the viewing is for monitoring the course of the disease state or identifying the diseased tissue for surgical intervention.

26. The method of claim 1, wherein the targeting construct is administered by a method selected from the group consisting of topically, intraarticularly, intracisternally, intraocularly, intraventricularly, intrathecally, intravenously, intramuscularly, intravascularly, intercavitarily, intraperitoneally, intradermally, and by a combination of any two or more thereof.

27. A method of performing a surgical procedure, the method comprising:
a) providing a light source device comprising one or more light sources configured to provide at least one excitation wavelength in the range from about 401 nm to about 510 nm or from about 470 nm to about 495 nm;
b) irradiating an in vivo body part of a subject containing diseased tissue with the light source and directly viewing fluorescence emitted in response to the light source, the fluorescence being emitted from a fluorescent targeting construct administered to the subject and which is specifically bound to or taken up by the diseased tissue in the body part;
c) determining a location or a surface area of the diseased tissue in the subject from the fluorescence provided by the targeting construct; and
d) removing at least a portion of the diseased tissue.

28. The method of claim 27, wherein the viewing of the fluorescence and the removing of the diseased tissue are performed substantially contemporaneously.

29. A light source device for performing the method of any of claim 1 or 27, the device comprising one or more light sources configured to provide at least one excitation wavelength in the range from about 401 nm to about 510 nm or from about 470 nm to about 495 nm.
